(19) United States
(12) Patent Application Publication
(10) Pub. No.: US 2010/0286150 A1
(43) Pub. Date: Nov. 11, 2010

(54) LEVOSIMENDAN FOR USE IN TREATING CHRONIC VALVULAR DISEASE

(76) Inventors: Fia Westerholm, Littoinen (FI); Jouko Levijoki, Littoinen (FI); Mira Korpivaara, Littoinen (FI); Minna Leppänen, Kangasala (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/523,228

(22) PCT Filed: Jan. 17, 2008
(86) PCT No.: PCT/FI08/00004

§ 371 Date: Sep. 16, 2009

(51) Int. Cl. A61K 31/50; A61P 9/00

(52) U.S. Cl. 514/247

(57) ABSTRACT

A method for the treatment of chronic valvular disease (CVD) in animals, particularly dogs, comprising administering to a subject in need thereof an effective amount of levosimendan or a pharmaceutically acceptable salt thereof. Levosimendan was shown to significantly reduce mortality and to significantly improve quality of life in dogs suffering from chronic valvular disease.

### TECHNICAL FIELD

The present invention relates to a field of veterinary medicine. In particular, the invention relates to a method for the treatment of chronic valvular disease (CVD) in animals, particularly dogs. The method comprises administering levosimendan or a pharmaceutically acceptable salt thereof to a subject in need of such treatment.

### BACKGROUND OF THE INVENTION

Chronic valvular disease (CVD), also referred to as myxomatous degenerative valve disease, is a common heart disease in dogs. It is characterized by a progressive degeneration and deformation of the atrioventricular valves, most commonly the mitral valves, resulting in early mitral valve insufficiency. This in turn leads to the appearance of a systolic heart murmur due to mitral regurgitation, wherein inadequate closure of the mitral valve causes blood to flow back to the left atrium. The affected dogs finally develop left atrioventricular volume overload, pulmonary edema, atrial dilatation and supraventricular arrhythmias.

Although dogs with CVD may exhibit good quality of life with standard therapy such as diuretics, ACE inhibitors and digoxin, the long-term prognosis is poor. Dogs may die suddenly from arrhythmias or a decision of euthanasia is made after severely worsened quality of life due to diuretic treatment failure. Mitral valve repair by surgical procedures is not readily available for animals. Thus, there is a need for improved veterinary therapies for reducing the risk of death in animals suffering from CVD.

Levosimendan, which is the (−)-enantiomer of [[4-(1,4,5,6-tetrahydro-4-methyl-6-oxo-3-pyridazinyl)phenyl]hydrazono]propanedinitrile, is an inotropic drug substance that is currently used as an intravenous infusion over the period of 24 hours for the short term treatment of human patients who suffer from acutely decompensated severe heart failure. The drug increases contractile force of the heart myocardium by enhancing the sensitivity of myofilaments to calcium. Levosimendan and a method for its preparation are described in U.S. Pat. No. 5,569,657.

### SUMMARY OF THE INVENTION

It has now been found that levosimendan or a pharmaceutically acceptable salt thereof is able to significantly reduce mortality, prolong survival and improve quality of life in animals, particularly dogs, suffering from chronic valvular disease. Levosimendan was effective and safe in the long-term oral treatment regimen in dogs making it particularly suitable for the veterinary treatment of chronic valvular disease.

Thus, the present invention provides a method for the treatment of chronic valvular disease (CVD) in animals, particularly dogs, comprising administering to a subject in need thereof an effective amount of levosimendan or a pharmaceutically acceptable salt thereof.

The present invention also provides a method for reducing mortality in animals, particularly dogs, suffering from chronic valvular disease (CVD), comprising administering to a subject in need thereof an effective amount of levosimendan or a pharmaceutically acceptable salt thereof.

The present invention also provides the use of levosimendan or a pharmaceutically acceptable salt thereof in the manufacture of a medicament for treating chronic valvular disease (CVD) in animals, particularly dogs.

The present invention also provides the use of levosimendan or a pharmaceutically acceptable salt thereof in the manufacture of a medicament for reducing mortality in animals, particularly dogs, suffering from chronic valvular disease (CVD).

The present invention also provides the use of levosimendan or a pharmaceutically acceptable salt in the manufacture of a medicament for improving quality of life in animals, particularly dogs, suffering from chronic valvular disease (CVD).

### BRIEF DESCRIPTION OF THE DRAWINGS

FIG. 1 shows the effect of 0.05 mg/kg of levosimendan (solid line) or placebo (dashed line) given orally twice a day during the period of 5 months on the mortality of dogs suffering from chronic valvular disease.

### DETAILED DESCRIPTION OF THE INVENTION

The term “chronic valvular disease” or “CVD” means herein a disease involving abnormality of one or more valve of the heart, particularly mitral and/or tricuspid valves, causing regurgitation. Thus, “chronic valvular disease” includes, for example, chronic degenerative valvular disease, myxomatous atrioventricular valvular degeneration, myxomatous mitral valve disease, chronic valvular fibrosis, mitral valve dysplasia, mitral regurgitation, tricuspid regurgitation, mitral valvular disease, mitral valve prolapse and endocardiosis.

The term “improving quality of life” means herein improving general well-being of an animal suffering from CVD, such improvement being apparent to the owner of the animal. The term includes reducing one or several symptoms of CVD, such as loss of appetite, exercise intolerance, daytime cough, nocturnal cough, and nocturnal restlessness.

The term “mg/kg of levosimendan or a pharmaceutically acceptable salt thereof” means milligram of levosimendan or a pharmaceutically acceptable salt thereof per one kilogram bodyweight of the subject to be treated, unless otherwise indicated.

The term “animal” means here non-human animals, such as non-human mammals. Variety of non-human mammals can be treated according to the present invention. According to one preferred embodiment of the invention, the mammal to be treated is a canine, feline, rodent, murine, equine, bovine or ovine species. According to one another preferred embodiment of the invention the mammal to be treated is a dog, cat or horse. According to a particularly preferred embodiment of the invention the mammal to be treated is a dog.

The terms “treating”, “treat” or “treatment” includes preventive (e.g. prophylactic) and palliative treatment.

According to the present invention levosimendan or a pharmaceutically acceptable salt thereof is administered to an animal in an amount effective for the treatment of chronic valvular disease (CVD). According to one embodiment of the invention, levosimendan or a pharmaceutically acceptable salt thereof is administered in an amount effective to ameliorate one or more of the symptoms of chronic valvular disease in an animal. According, to further embodiment of the invention, levosimendan or a pharmaceutically acceptable salt thereof is administered in an amount effective to reduce mortality in an animal suffering from chronic valvular disease (CVD). According to further embodiment of the invention, levosimendan or a pharmaceutically acceptable salt thereof is administered in an amount effective to improve quality of life in an animal suffering from chronic valvular disease (CVD).

According to one embodiment of the invention, levosimendan or a pharmaceutically acceptable salt thereof is used to treat chronic valvular disease in animals, particularly dogs, with preserved myocardial contractility.

According to one embodiment of the invention, levosimendan or a pharmaceutically acceptable salt thereof is used to reduce mortality caused by chronic valvular disease in animals, particularly dogs, with preserved myocardial contractility.

Chronic valvular disease (CVD) can be diagnosed by known methods including physical examination, echocardiography and radiology. Evident systolic murmur over valvular area is a typical feature of chronic valvular disease.

The administration of levosimendan or a pharmaceutically acceptable salt thereof to the animal can be by e.g. oral, parenteral, transmucosal or transdermal route. For the long-term treatment of chronic valvular disease, oral administration is particularly preferred.

In general, levosimendan or a pharmaceutically acceptable salt thereof can be administered orally to an animal in a daily dose suitably ranging from about 0.005 to about 0.3 mg/kg, for example from 0.01 to 0.2 mg/kg depending on the age, weight, condition and the species of the animal. According to one particularly preferred embodiment of the invention, levosimendan or a pharmaceutically acceptable salt thereof is administered orally to an animal, particularly a dog, in a daily dose ranging from about 0.03 to about 0.15 mg/kg, for example from about 0.07 to 0.12 mg/kg.

If intravenous administration is desired, levosimendan or a pharmaceutically acceptable salt thereof can be administered by intravenous infusion using the infusion rate from about 0.01 to 5 μg/kg/min, more typically from about 0.02 to 3 μg/kg/min.

The active ingredient of the invention may be administered daily or several times a day or periodically, e.g. weekly or biweekly, depending on the condition of the animal to be treated. Normally, daily administration, e.g. two times daily, is preferred when the active ingredient is administered orally.

Levosimendan or a pharmaceutically acceptable salt thereof may be administered alone or together with other therapeutic agents suitable in the treatment of chronic valvular disease.

Levosimendan or a pharmaceutically acceptable salt thereof is formulated into dosage forms using principles well known to practitioners in the art. It is given to a patient as such or preferably in combination with suitable pharmaceutical excipients in the form of tablets, granules, capsules, suppositories, emulsions, suspensions or solutions whereby the contents of the active compound in the formulation is from about 0.1 to about 100% per weight. Choosing suitable ingredients for the composition is routine for those of ordinary skill in the art. It is evident that suitable carriers, solvents, gel forming ingredients, dispersion forming ingredients, antioxidants, colours, sweeteners, flavouring agents, wetting agents, release controlling components and other ingredients normally used in this field of technology also may be used.

For oral administration in tablet form, suitable carriers and excipients include e.g. lactose, corn starch, magnesium stearate, calcium phosphate and talc. For oral administration in capsule form, useful carriers and excipients include e.g. lactose, corn starch, magnesium stearate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil (sold under trade name Cutina HR), cotton seed oil (sold under the trade names Sterotex or Lubritab) or a mixture thereof; fatty acid esters such as triglycerides of saturated fatty acids or their mixtures e.g. glyceryl tristearates, glyceryl tripalmitates, glyceryl trimyristates, glyceryl tribehenates (sold under the trade name Compritol) and glyceryl palmitostearic acid ester.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule for the treatment chronic valvular disease in dog comprises from about 0.1 to 2 mg, more typically 0.2 to 1 mg, of levosimendan or a pharmaceutically acceptable salt thereof.

Formulations suitable for intravenous administration such as injection or infusion formulation comprise sterile isotonic solutions of levosimendan or a pharmaceutically acceptable salt thereof and vehicle, preferably pharmaceutically acceptable aqueous solutions.

Typically an intravenous infusion solution comprises from about 0.001 to 1, preferably from about 0.01 to 0.1 mg/ml, of levosimendan or a pharmaceutically acceptable salt thereof. The formulation for intravenous administration may also be in the form of an infusion concentrate, which is diluted with an aqueous vehicle before use. Typically such infusion concentrate comprises levosimendan or a pharmaceutically acceptable salt thereof dissolved in dehydrated ethanol.

Salts of levosimendan may be prepared by known methods. Pharmaceutically acceptable salts are useful as active medicaments, however, preferred salts are the salts with alkali or alkaline earth metals.

### Example 1

A double-blind placebo-controlled study was conducted for evaluating the long-term efficacy and safety of levosimendan and its effect on long-term survival in dogs diagnosed with chronic valvular disease (CVD). Dogs were randomised to receive either 0.05 mg/kg of levosimendan (n=40) or placebo (n=40) orally twice a day for 5 months. All dogs were allowed to receive their background therapy (ACE-inhibitors, diuretics, beta-blockers and/or digoxin). Quality of life, symptoms of CVD, safety and mortality assessment were conducted throughout the study.

Improvement in quality of life was determined by using a composite owner-reported symptom score (ORSS). ORSS is a composite variable that was calculated by summing scores for appetite, intolerance to exercise, daytime cough, nocturnal cough, and nocturnal restlessness. The owner-reported symptom score could range from 0 (best possible) to 18 (worst possible).

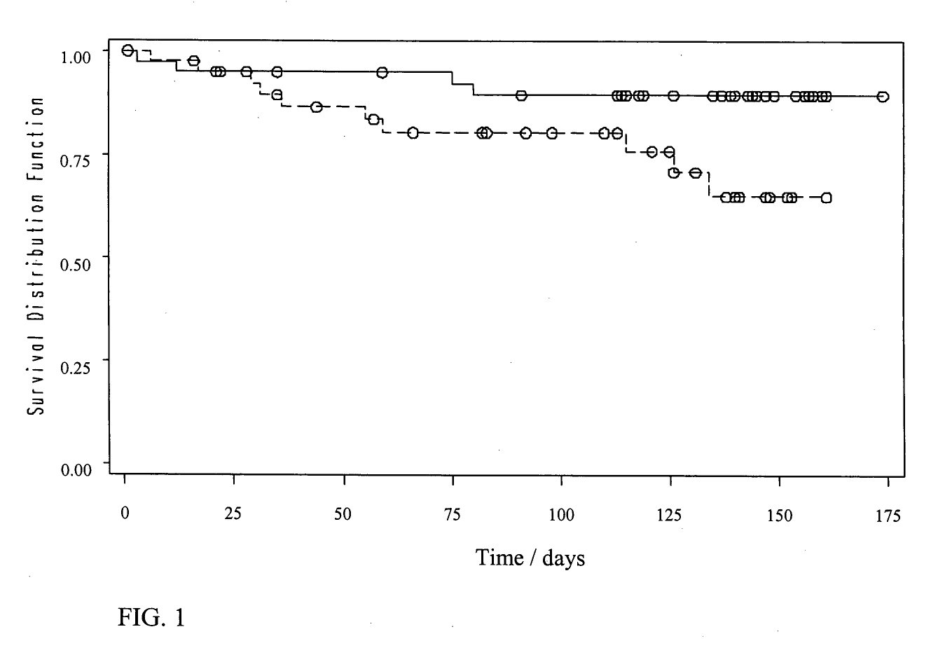

The survival of the dogs in the levosimendan group (solid line) and the placebo group (dashed line) during the treatment period is shown in FIG. 1. Dogs that were alive but withdrawn or censored from the study are shown as spheres. It can be seen that addition of oral levosimendan to the standard therapy significantly reduced mortality and prolonged survival in dogs suffering from chronic valvular disease (CVD). Mean owner-reported symptom score over time for each treatment group is summarized in Table 1. Compared to baseline assessments (0 months), quality of life significantly improved over time for the levosimendan group (group A), but not for the placebo group (group C).

No significant safety concerns were identified in the levosimendan group.

TABLE 1
Owner-reported symptom score over time (mean ± SD)

| Treatment | Time (months) | | | |
|---|---|---|---|---|
| group | 0 | 1 | 3 | 5 |
| A | 5.6 ± 2.4 | 4.1 ± 3.2 | 4.4 ± 4.1 | 4.4 ± 3.4 |
| C | 5.4 ± 2.2 | 5.2 ± 3.3 | 6.7 ± 4.9 | 7.0 ± 4.2 |
| (placebo) | | | | |

1. A method for the treatment of chronic valvular disease (CVD) in animals, comprising administering to a subject in need thereof an effective amount of levosimendan or a pharmaceutically acceptable salt thereof.

2. The method according to claim 1, wherein levosimendan or a pharmaceutically acceptable salt thereof is administered orally.

3. The method according to claim 1, wherein levosimendan or a pharmaceutically acceptable salt thereof is administered in a daily amount ranging from 0.03 mg/kg to about 0.15 mg/kg.

4. A method for reducing mortality in animals, suffering from chronic valvular disease (CVD), comprising administering to a subject in need thereof an effective amount of levosimendan or a pharmaceutically acceptable salt thereof.

5. The method according to claim 4, wherein levosimendan or a pharmaceutically acceptable salt thereof is administered orally.

6. The method according to claim 4, wherein levosimendan or a pharmaceutically acceptable salt thereof is administered in a daily amount ranging from 0.03 mg/kg to about 0.15 mg/kg.

7. A method for improving quality of life in animals, suffering from chronic valvular disease (CVD), comprising administering to a subject in need thereof an effective amount of levosimendan or a pharmaceutically acceptable salt thereof.

8-10. (canceled)

11. The method according to claim 1, wherein the subject is a dog.

12. The method according to claim 4, wherein the subject is a dog.

13. The method according to claim 7, wherein the subject is a dog.
